(19) United States
(12) Patent Application Publication
(10) Pub. No.: US 2010/0061888 A1
(43) Pub. Date: Mar. 11, 2010

(54) MAGNETICALLY MODIFIED AEROSOL DECONTAMINATION APPARATUS AND METHOD

(75) Inventors: Paul Stolz, Plymouth, MI (US); Ralph M. Sias, Oceanside, CA (US); Mark A. Hale, Canton, MI (US); Jeff Szekely, Morrow, OH (US)

Correspondence Address: BROOKS KUSHMAN P.C., 1000 TOWN CENTER, TWENTY-SECOND FLOOR, SOUTHFIELD, MI, 48075, US

(73) Assignee: ADVANCED DISINFECTION TECHNOLOGIES, LLC, Troy, MI (US)

(21) Appl. No.: 12/484,689

(22) Filed: Jun. 15, 2009

Related U.S. Application Data
(60) Provisional application No. 61/191,250, filed on Sep. 8, 2008.

(51) Int. Cl. A61L 2/22

(52) U.S. Cl. 422/28; 422/292

(57) ABSTRACT

Decontamination is achieved by practicing a method using the disclosed apparatus for producing a magnetically energized, excited decontamination aerosol 26. The apparatus has a source of decontamination fluid 10 and an aerosol producer 12 that operates at substantially one atmosphere ambient pressure. A magnetic energizer 20 modifies the energy state of the aerosol 14 which then passes through a charging ring 24. The further modified, excited droplets 26 then permeate a location or contact an object 28 to be decontaminated.

### CROSS-REFERENCE TO RELATED APPLICATION

This application claims the benefit of U.S. application Ser. No. 61/191,250 filed Sep. 8, 2008, the disclosure of which is incorporated herein by reference.

### BACKGROUND OF THE INVENTION

1. Field of the Invention

The invention relates to an apparatus and method for decontaminating an object or environment using a magnetically modified aerosol.

2. Background Art

A recent outbreak of Swine flu has heightened concerns and has renewed interest in cost-effective remedial decontamination measures:
“The threat of deadly new viruses is on the rise due to population growth, climate change and increased contact between humans and animals. What the world needs to do to prepare . . . Today we remain underprepared for any pandemic or major outbreak, whether it comes from newly emerging infectious diseases, bioterror attack or laboratory accident . . . . In our lifetimes, or our children's lifetimes, we will face a broad array of dangerous emerging 21^{st}-century diseases, man-made or natural, brand-new or old, newly resistant to our current vaccines and antiviral drugs . . . . World-wide access to infectious agents and basic biological know-how has grown more rapidly than even the exponential growth of computing power . . . . Over the last decades, we have seen more than three dozen new infectious diseases appear, some of which could kill millions of people with one or two unlucky gene mutations or one or two unfavorable environmental changes. The risks of pandemics only increase as the human population grows, the world loses greenbelts, uninhabited land disappears and more humans hunt and eat wild animals.” L. Brilliant, WSJ, pp. W1-W2 (May 2-3, 2009).

Elimination and containment of adverse microbiological species are relevant to environmental control and damage containment in a peacetime or warfare setting. To avoid contamination, drugs and medical devices may be cleaned and encased in sterile packaging. In a hospital, operating rooms, wards, and examination rooms may be sterilized so that injurious microbiological organisms cannot spread from one patient to another. Wounds can be cleansed to prevent infection. But such measures entail time and money.

Biological warfare and bioterrorism may involve injurious microbiological organisms that are deliberately released as widely as possible in such a way as to wreak havoc. Even small amounts of certain microbiological organisms may achieve widespread contamination. Some microbiological organisms can lie in a dormant state before becoming active. Such situations challenge those who wish to control and eradicate the assault.

Certain approaches for controlling the spread of microbiological organisms have been developed for relatively small-scale use in well-controlled environments and where the risk of propagation is small. But such approaches are of limited value in combating biological warfare and bioterrorism. Fresh approaches are needed in contaminated environments. The present invention fulfills this need, and further provides related advantages.

Illustrative of related prior art is U.S. Pat. No. 7,008,592 which discloses the use of an aerosol of reactive oxygen species that are charged by an electrical or photonic source (“592 patent, 5:60) into a plasma, ion or free radical state. The '592 patent purports to be an improvement over prior art systems which need a magnetic or electrostatic energy source, such as U.S. Pat. No. 5,750,072 (Sangster) and U.S. Pat. No. 4,704,942 (Barditch).

### SUMMARY OF THE INVENTION

Broadly stated, one embodiment of the invention includes a decontamination apparatus for killing microorganisms on contact using a highly active aerosol mist, the microorganisms being situated on a substrate or suspended in an ambient environment.

The apparatus comprises:

A. a source of a decontamination fluid;

B. an aerosol producer for receiving the decontamination fluid and creating an aerosol of uncharged droplets;

C. a magnetic energizer through which the aerosol passes, the energizer including a high density, uniform array magnetic field to modify the energy state of the droplets and create reactive oxygen species in or on the surface of the droplets, thereby creating modified droplets; and

D. a charging ring that receives the modified droplets, aligns ions therewithin and moves reactive oxygen species towards the surface of the droplets, thereby creating further modified droplets and allowing the further modified droplets to remain in a stable form in transit to the substrate or environment to be treated.

The further modified droplets may penetrate an area to be treated without dependence solely on forced airflow because they are small, are similarly charged and thus mutually repulsive. This causes them readily to diffuse into the area being treated.

Preferably, the further modified droplets react at an ambient pressure and temperature with contaminants associated with the substrate or environment to be treated. They are transformed in situ into an uncharged state, thus decontaminating the substrate or environment. One or more benign reaction products are created that leave the substrate undamaged. They volatilize or optionally may leave a bio-protective film on the substrate.

In one approach, a method for decontaminating a microorganism situated on a substrate or suspended in an ambient environment, includes, in general, the steps of:

I. providing a source of a decontamination fluid;

II. introducing an aerosol producer that receives the decontamination fluid and creates an aerosol of uncharged droplets;

III. propelling the aerosol of uncharged droplets through a magnetic energizer, thereby subjecting them to a high density, substantially uniform array magnetic field to modify the energy state of the droplets and create reactive oxygen species in or on the surface of the droplets, thereby creating modified droplets; and

IV. passing the modified droplets through a charging ring that aligns ions therewithin and moves reactive oxygen species towards the surface of the droplets.

The invention, therefore, includes an apparatus and method for decontaminating environments and articles from adverse microbiological organisms. Decontamination occurs rapidly, and often on contact between the organism and a charged droplet that has reactive oxygen species aligned on the droplet surface. Because the charged droplets are small (1-100 microns in diameter) a line of sight to the contaminated region is not required, so that the microbiological organisms cannot escape destruction by being reposed in remote locations.

The disclosed system may be readily scaled from small to large sizes of apparatus and decontaminated regions for use in civilian and military applications. It may be used (1) within enclosures to decontaminate articles or, for example, hands; (2) to decontaminate enclosed spaces, such as rooms and ventilating systems; (3) in open spaces to decontaminate entire areas.

Decontamination may occur without persistent chemicals that otherwise may be toxic and cause harm, or leave unwanted residues. Decontamination operates by a chemical reaction that is benign—it does not cause mutation of the biological microorganism toward a decontamination-resistant strain. Without wishing to be bound by any particular theory, it is believed that a benefit of an oscillating magnetic field, for example, is that a change is induced in the bacterial, fungal and viral molecular structure by the field's energy. When the microorganisms are transformed into a vulnerable condition, they are more subject to the effect of the excited decontamination fluid. Alternatively, a thin film may be left on the decontaminated surfaces of a persistent chemical in various non medical applications, such as pest control or mold remediation.

One aspect of the present system is that the apparatus and method can, but need not, operate at substantially one atmosphere pressure in the ambient environment; i.e., in some situations the apparatus itself may create positive pressure to urge the aerosol mist from the apparatus. Nevertheless, the environment in which the apparatus operates is virtually at atmosphere pressure. In contrast, some prior decontamination systems operate in a vacuum. Although that may be useful in sterilizing objects that may be put into a low pressure chamber, it is impractical for decontaminating objects in areas that cannot be evacuated.

The disclosed apparatus and method offer an effective way to combat microbiological organisms. It is effective in enclosed area and open spaces. It is therefore effective in many situations where biological microorganisms have been intentionally spawned over wide areas or have intentionally propagated.

Optionally, the disclosed techniques can be practiced with a controller or other microprocessor.

Other features and advantages of the present invention will be apparent from the following more detailed description of the preferred embodiment, taken in conjunction with the accompanying drawings, which illustrate, by way of example, the principles of the invention. The scope of the invention is not, however, limited to this preferred embodiment.

### BRIEF DESCRIPTION OF THE DRAWINGS

FIG. 1 is a schematic view of a first embodiment of apparatus for practicing the invention; and

FIG. 2 is a block flow diagram of a preferred system for practicing the invention.

### DETAILED DESCRIPTION OF THE PREFERRED EMBODIMENT(S)

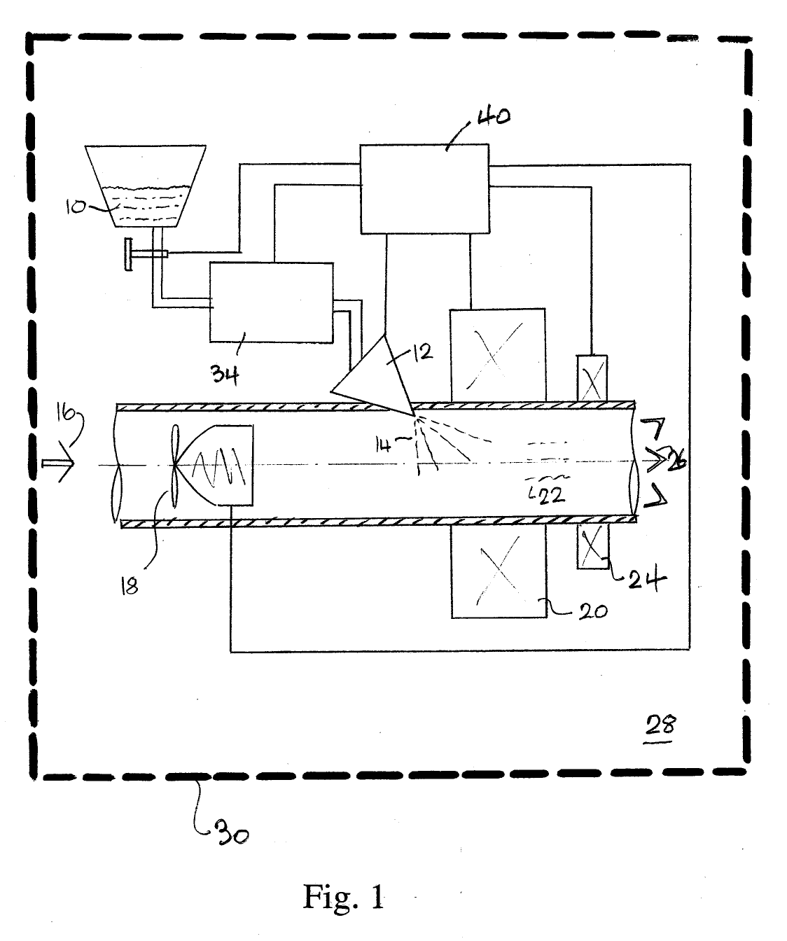

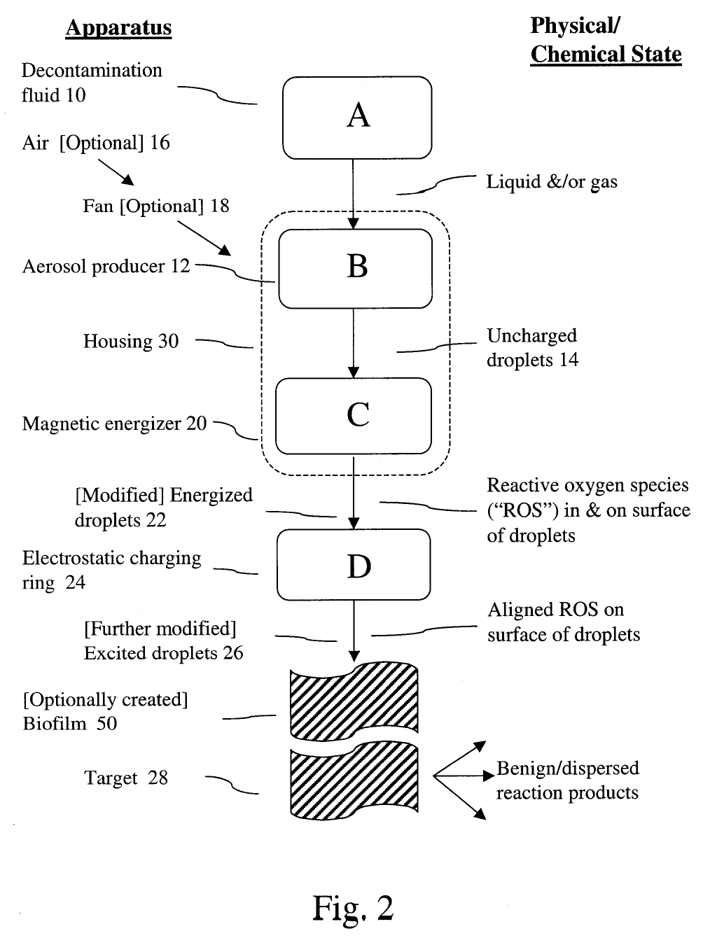

FIG. 1 depicts one illustrative apparatus configuration for performing decontamination, and FIG. 2 is a flow diagram that depicts the physical and chemical states of fluids as they transit therethrough.

The apparatus comprises:

A. a source of a decontamination fluid;

B. an aerosol producer for receiving the decontamination fluid and creating an aerosol of uncharged droplets;

C. a magnetic energizer through which the aerosol passes, the energizer including a high density, uniform array magnetic field to modify the energy state of the droplets and create reactive oxygen species in or on the surface of the droplets, thereby creating modified droplets; and

D. a charging ring that receives the modified droplets, aligns ions therewithin and moves reactive oxygen species towards the surface of the droplets, thereby creating further modified droplets and allowing the further modified droplets to remain in a stable form in transit to the substrate or environment to be treated.

The further modified droplets penetrate an area to be treated without dependence on airflow because they are small, are similarly charged and thus mutually repulsive. This causes them readily to diffuse into the area be treated.

Preferably, the further modified droplets react at an ambient pressure and temperature with contaminants associated with the substrate or environment to be treated. They are transformed in situ into an uncharged state, thus decontaminating the substrate or environment. One or more benign reaction products are created that leave the substrate undamaged. They volatilize or optionally may leave a bio-protective film on the substrate.

In one approach, a method for decontaminating a microorganism situated on a substrate or suspended in an ambient environment, includes, in general, the steps of:

I. providing a source of a decontamination fluid;

II. introducing an aerosol producer that receives the decontamination fluid and creates an aerosol of uncharged droplets;

III. propelling the aerosol of uncharged droplets through a magnetic energizer, thereby subjecting them to a high density, substantially uniform array magnetic field to modify the energy state of the droplets and create reactive oxygen species in or on the surface of the droplets, thereby creating modified droplets; and

IV. passing the modified droplets through a charging ring that aligns ions therewithin and moves reactive oxygen species towards the surface of the droplets.

The decontamination fluid preferably comprises three or more components (A-C). There is 0.1%-10% of component A which is selected from the group consisting of hydrogen peroxide, urea peroxide and other organic peroxides. Component B is present in an amount of 70%-98% and is selected from the group consisting of water and de-ionized water. Component C may be present in an amount of 1%-10%, and is selected from the group consisting of isopropyl alcohol propyl alcohol, ethyl alcohol and protic/aprotic polar solvents. A two part decontamination fluid of A and B may still function, but A-B-C is preferred.

If desired, the starting fluid may also include boric acid that has been found to be helpful in treating a substrate or an environment that includes an infestation of bed bugs, lice and like pests. Also, if desired, the decontamination fluid may include ozone. It has been found that this chemical can aid in the creation of reactive oxygen species.

The decontamination fluid 10 is preferably a liquid that may be vaporized in ambient-pressure air by an aerosol producer 12 to form an aerosol of uncharged droplets 14. In liquid form, the decontamination fluid 10 maybe stored at one atmosphere or a slightly greater pressure. If in a gaseous state, the decontamination fluid 10 may require pressurized storage. The source of the decontamination fluid may also be a precursor of the decontamination fluid, such as a solid, liquid, or gas. Suitable decontamination fluids are preferably aqueous solutions. Alternatively, they may be (1) solutions in organics such as alcohol; or (2) a source of a decontamination fluid precursor that chemically reacts or decomposes to produce the decontamination fluid.

Preferably, air 16 driven by a fan 18 can be used as a propellant.

Suitable decontamination fluids contain one or more magnetically excitable species, e.g., a reactive oxygen specie that has hydroxyl ions (OH⁻) for subsequent excitation. Such a source may be hydrogen peroxide (H₂O₂) or a precursor specie that produces hydroxyl ions. Hydrogen peroxide is a preferred starting fluid. It is effective in rapidly overcoming many types of biological microorganisms, is normally available in an aqueous solution, decomposes eventually to oxygen and water, leaves no chemical residue after decomposition, is nontoxic and harmless to man and animals in its original and decomposed forms, is cheap and readily available. Other sources of hydroxyl ions include peracetic acid (CH₃COOOH), sodium percarbonate (Na₂CO₃-1.5HOH), and glutaraldehyde (OCH(CH₂)₃CHO).

The initial decontamination fluid may also contain 0.05-3% of promoting species that are not themselves sources of energizable species such as hydroxyl ions, but instead influence the decontamination reactions. Examples include chelated metal ions and ethylenediaminetetraacetic acid (EDTA), which binds metal ions and allows the activated species to destroy the cell walls more readily; an alcohol such as isopropyl alcohol, which improves wetting of the mist to the cells; enzymes, which speed up or intensity the redox reaction in which the activated species attacks the cell walls; fatty acids, which act as an ancillary anti-microbial and may combine with free radicals to create residual anti-microbial activity; and acids such as citric acid, lactic acid, or oxalic acid, which speed up or intensity the redox reaction and may act as ancillary anti-microbial species to pH-sensitive organisms. Mixtures of various excitable species and promoting species may optionally be used.

Optionally, an antimicrobial is added to the decontamination fluid that may leave a bioprotective residue on the substrate after decontamination.

The aerosol producer 12 may be any device that generates a mist 14 of the decontamination fluid 10. Illustrative embodiments include a fogger, a nebulizer and a spray nozzle. One suitable aerosol producer is available from Ocean Mistg. An illustrative embodiment is the ZS-30 ultrasonic humidifier. In one series of experiments, the ZS-30 model served as an ultrasonic nebulizer inside a fogger that generated 40 ml/min up to 175 ml/min (about 90 ml/min preferred). Optionally, a smaller aerosol producer can be used if the disinfection system is used with a chamber. In such situations, a range of 15 ml/min up to 60 ml/min are suitable output flow rates (with about 35 ml/min being preferred). If the disinfection system is embodied in a hand unit, corresponding performance data include a 1 ml/min-10 ml/min output volume (about 7 ml/min being preferred).

The aerosol producer 12 may instead be a spray head such as a high-pressure spray head that establishes ultrasonic waves in the uncharged droplets.

Optionally, the aerosol producer 12 produces a pressure of the mist 14 that is above one atmosphere upon emergence from the aerosol producer. Such pressure may help distribute the droplets into an ambient environment. Such emergent overpressure before distribution of the droplets into a magnetic energizer 20 or a charging ring 24 and the environment to be decontaminated at one atmosphere pressure is considered within the term “substantially one atmosphere ambient pressure”.

Upon emergence from the aerosol producer 12, a decontamination fluid mist 14, preferably of uncharged droplets contains excitable species and optionally promoting species. In the preferred case, the aerosol 14 includes fine droplets of the vaporized decontamination fluid. The droplets are preferably roughly uniformly sized, from about 1 to about 100 microns in diameter.

If two or more decontamination fluids 10 or components are used, they may be mixed together and vaporized in a single aerosol producer 12. However, if the components of the decontamination fluid 10 are not compatibly vaporized, and a separate aerosol producer 12 can be provided for each fluid source. A commercial aerosol generator is typically tuned for the specific fluid to be vaporized, so that optimal vapor production occurs only for that specific fluid or closely similar fluids. If multiple decontamination fluids or components of decontamination fluids are used with substantially different fluid and vaporization properties, it is usually necessary to provide a separate aerosol producer 12 for each of the flows of decontamination fluid. The disclosure herein of an aerosol producer 12 in relation to the subsequently discussed embodiments includes both single and multiple aerosol producers used in combination.

As noted above, the decontamination fluid aerosol 14 of uncharged droplets is energized by a magnetic energizer 20 to produce modified or energized droplets 22 in an ionized, plasma, or free radical state. After passage through the charging ring 24, almost all of the droplets 22 are further modified and excited to create further modified or excited droplets 26, together with the promoting species, if any are present. A high yield of modified species is desirable to improve the efficiency of the decontamination process.

The energizing field in the energizer 20 is preferably magnetic. The magnetic energizer 20 receives a 12-24 volt direct current (that creates a constant magnetic field), or a (preferably) 5-120 volt alternating current (that creates an oscillating magnetic field; low frequencies are preferred—up to 1 KHZ), which produces a 3-40 amp current flow and a flux density of 300-1000 Gauss (800-1000 Gauss is preferred). Uncharged droplets 14 become energized upon passing through the magnetic energizer 20 during a transit time that is between about 0.5-3 seconds. Over that time, all molecules of the uncharged droplets 14 are activated to some degree. About 20-80% of the decontamination fluid 10 (for example, hydrogen peroxide) in each droplet is energized. Suitable embodiments of magnetic energizers may be constructed from a Helmholtz coil (preferred), a Maxwell coil, and other such coil arrays that are energized by a direct or alternating electric current. Details of such coils are found, for example, at http://en.wikipedia.org/wiki/Helmholtz_coil and http://en.wikipedia.org/wiki/Maxwell_coil. Each is incorporated by reference.

Other things being equal, a Maxwell coil tends to generate a higher density of magnetic flux. However, the magnetic field generated tends not to be as uniform as that provided by a Helmholtz coil. In some circumstances, a Maxwell coil may be preferred if it is desired to energize a small stream of aerosol, such as might be delivered along the tube having an inside diameter of about ¼″. In contrast, the Helmholtz coil can be adapted to receive larger tubes which are then susceptible to a relatively homogeneous magnetic field.

Generally, the inside diameters of suitable Helmholtz coils range from 0.5″-6″, 3″-4″ being preferred. In one experiment, using an ERSE 13 millihenry A/C inductor, a current flow of 10-12 amps through a pair of 12 gauge air core inductors was created under a potential difference of 24 volts.

The nature of Helmholtz coils requires that the coils be placed approximately one radius apart. If an object to be decontaminated needed to be placed within the coils, this would limit the volume in which an object could be sterilized.

The magnetic field created by a Helmsholtz coil is significantly reduced as the diameter of the coil increases, as shown in the formula:

H≈0.899 times NI/R,

where:
H is the magnetic field in oersteds,
N is the number of turns per coil,
I is the coil current in amperes, and
R is the coil radius in centimeters.

Preferably, the aerosol is propelled by an air flow through an array of Helmholtz coils that have a large number of winding and a relatively small diameter. It will be appreciated that the ratio of the number of windings to the radius of the coils directly affects the density of the magnetic field created by the coils. A small diameter and a large number of windings tend to create a stronger magnetic field. Placing the coils one radius apart focuses the magnetic field along the center axis of the array.

The Helmholtz coil array is typically powered by a variable voltage alternating current supply that allows the decontamination system to be tuned for maximum efficiency. The Helmholtz array can be coupled with a variable power supply using a suitable capacitive cupler to prevent overheating when using larger current flows. Non-polarized electrolytic capacitor of 100 μf have been found to produce current flows of 2.4 to 3 amps. This allows a strong magnetic field to be generated and focused while producing little heat in the coils or the capacitor.

Upon emergence from the magnetic energizer 20, the energized droplets 22 house reactive oxygen species within and on the surface of the droplets. The energized species 22 then pass through the electrostatic charging ring 24. The charging ring 24 aligns ions within the energized droplets and moves reactive oxygen species toward the surface of the droplets, thereby further modifying the energized droplets to form excited droplets 26. This allows most of the further modified droplets 26 to remain in a relatively stable form, in which they may be held in transit to the target substrate 28 or environment to be treated.

In one example, the size of the charging ring 24 approximated the inside diameter of a Helmholtz coil. Aluminum is a preferred conductor that is used in the charging ring 24. Stainless steel is an alternative. One example of a suitable charging ring is the EMCO G10 high voltage converter. In one experiment, the current flow through the charging ring 24 was 0.1-0.6 milliamps. Preferably, the charging ring is annular and its inner diameter is circumscribed by 90° edges that are sharply defined.

Without wishing to be bound by a particular theory, the excited droplets 26 then enter redox reactions with the cell walls of the microbiological organisms, thereby destroying the cells or at least preventing their multiplication and growth. If the decontamination fluid 10 includes hydrogen peroxide (even in diluted form), at least some of the H₂O₂ molecules dissociate to produce hydroxyl (OH⁻) and monatomic oxygen (O⁻) ionic excited species. These species remain dissociated for a period of time, typically several seconds or longer, during which they attack and destroy the biological microorganisms. In the case of hydrogen peroxide, the dissociated ionic species recombine to form harmless diatomic oxygen and water, thereby leaving the cleansed environment undamaged.

The magnetically modified aerosol uses a class of antimicrobial/sporicidal agent, preferably such as hydrogen peroxide (H₂O₂). Antimicrobials such as H₂O₂ kill microorganisms because they slowly dissociate creating Reactive Oxidative Species (ROS). The ROS react with spore walls, cell walls, cell cytoplasm, cell nuclei, and DNA to kill the microorganism through lysing (i.e., physically breaking open the cell). Such reactions are known as “redox” reactions and are recognized to physically destroy microorganisms, preventing mutations that can lead to drug-resistant strains. The magnetically modified aerosol significantly increases the available oxidative species, thereby enhancing the redox reaction.

Other sterilization systems, such as radiation and non-redox based chemicals do not lyse cell walls and instead attack a specific part of the microorganism's life cycle. For example, radiation (gamma and electron beam) breaks the ends of DNA strands. Alcohol interrupts the cell's osmotic cycle. Resistant microorganisms can survive non-redox based systems because they are not physically destroyed and can therefore mutate into resistant strains.

In the embodiment of FIG. 1, an energizer 20, schematically illustrated as a magnetic coil, through which the decontamination fluid aerosol 14 passes, is located proximate to, and preferably immediately adjacent to, the aerosol producer 12. The aerosol producer 12 and the magnetic energizer 20 may be juxtaposed so that the energizer 20 modifies the uncharged droplets 14 as they leave the aerosol producer 12. Alternatively, the magnetic energizer 20 may be located remotely from the aerosol producer 12, so that the mist 14 is generated to fill a space and is then modified.

The aerosol producer 12 and the magnetic energizer 20 are typically packaged together for convenience in a single housing 30 (FIG. 2). The decontamination fluid aerosol 14 leaving the aerosol producer 12 is preferably immediately energized by the magnetic energizer 20. The decontamination fluid aerosol 14 flows from the aerosol producer 12 and remains as a non-activated decontamination fluid mist of uncharged droplets prior to passing under the influence of and being energized by the magnetic energizer 20. Emergent modified droplets 22 are further excited by the charging ring 24 to produce excited droplets 26 of decontamination fluid aerosol.

Turning now to FIG. 2, it will be seen that for illustrative purposes only, the various apparatus components A-D are depicted in a sequence of A-D. It should be recognized that the apparatus of the disclosed invention is not so limited. For example, other physical arrangements are contemplated. They include A, C, D, B and A, C, B, D.

In practicing the disclosed method and apparatus, the system results in further modified, excited droplets 26 that have an average particle size of 1-100 microns to facilitate penetration into the area to be treated. Dispersion is relatively widespread without significant dependence on airflow. Because the excited droplets 26 are similarly charged and therefore mutually repulsive, they readily diffuse into an ambient environment or onto a substrate to be treated. Thus, the decontamination apparatus may be used to decontaminate air and other gas flows, in addition to solid objects.

For some applications, the apparatus may include a chamber 30 or other enclosed space into which the further modified, excited aerosol decontamination fluid 26 is directed by the magnetic energizer 20 and charging ring 24. The chamber 30 may receive objects 28 to be decontaminated. Optionally, urged by a fan 18, it may receive a moving volume of a contaminated gas such as air to be purified. As noted, the chamber 30 may define an enclosed space such as a room or an interior of a vehicle, which is to be decontaminated. But use of the invention is not so limited. There may be no chamber 30, in which case the excited aerosol droplets 26 are propelled into free space to treat an unenclosed, open area. The decontamination method of choice is effective in various environments to destroy biological microorganisms, although it is most efficient when constrained by enclosures and pre-defined areas.

One concern with biowarfare microorganisms is that they are air-borne, and are transmitted from one area to another by flows of air. In a building or vehicle, once the microorganisms have entered the HVAC (heating, ventilating, and air conditioning) system in one room, they may be conveyed quickly to another part of the building. The microorganisms contaminate the entire building and the HVAC ducting, so that major cleanup efforts are required. A virtue of the present approach is that the decontamination mist is also air-borne, and readily mixes with the air-borne microorganisms to attack them.

All of these modes of deployment preferably operate in an ambient pressure of about one atmosphere or slightly above one atmosphere, all of which as noted above are within the scope of “substantially one atmosphere ambient pressure”. Accordingly, the system does not require vacuum chambers or pressure chambers. The aerosol producer 12 may produce a slight overpressure of the aerosol 14 as it enters the one-atmosphere environment, but does not require either a vacuum or a pressure chamber. The present approach is operable in other environments, such as less than or more than one atmosphere pressure, but does not require such higher or lower pressures to be functional.

It is contemplated that one embodiment of the disclosed system will include a programmable controller or microprocessor 32 (FIG. 1) with system monitoring to allow for room profiles to be pre-loaded. This will significantly improve the ability to kill on all open surfaces in a hospital room while speeding the treatment cycle and faster turnaround of the room.

As depicted in FIG. 1 a controller 40 is actuated by a start cycle instruction. Such a controller may be a PLC manufactured by Siemens. In one embodiment, the controller 40 interrogates the decontamination fluid supply 10 to confirm the presence of fluid 10. The controller 40 then activates an AC power supply and a DC power unit which activates the fan 18 and the aerosol producer 12. If desired, the controller 40 may interrogate the aerosol producer 12 to check its operational state. The controller 40 can modulate a level control valve 34 to insure that the aerosol producer 12 has the proper volume of fluid 10.

The controller 40 then optionally may activate the aerosol producer 12 to generate an aerosol 14 into the airstream 16 provided by the fan 18. The controller 40 may also activate the variable voltage AC power supply and the magnetic energizer 20 to generate the modified energized droplets 22 in the flow.

The magnetic energizer 20 is preferably powered by a variable voltage AC supply to allow tuning of the system for maximum efficacy. The energizer 20 may be coupled with the variable power supply using a suitable capacitive coupler to prevent overheating while using larger current flows. Non-polarized electrolytic capacitors of 100 μf have been found to produce current flows of 2.4 to 3 amps. This allows a strong magnetic field to be generated and focused, while producing little heat in the coils or capacitor.

The object 28 being treated is exposed to the further modified, excited aerosol 26 as it exits the magnetic energizer 20 and charging ring 24. The object 28 should be reasonably proximate to the coils, preferably within a normal distance of about 18 inches.

The cycle is terminated when the controller 40 receives a stop signal, at which point it shuts down all of the apparatus components.

A number of tests of the present approach were performed, and some representative results are now discussed.

### EXAMPLES

In an exemplary use, the room width, height and length were measured in order to determine the volume of the space to be decontaminated. Efficacy is a function of flow rate, volume of space to be treated, and the desired efficacy level. In general, efficacy is directly related to the dose that is administered to a predefined volume.

A. In one series of experiments, an aerosol dosing rate for open rooms with an embodiment of the subject decontamination equipment ranged between 0.2 ml/ft³ and 0.75 ml/ft³. Efficacy levels between 1 log and >6 log can be achieved by increasing the dose per cubic foot.

By experiment, it has been found that the treatment cycle time can be estimated by the following formula:

Treatment time [min]=Aerosol dose volume [ml/ft³]×volume of space to be decontaminated [ft³]/aerosol flow rate [ml/min]

For instance, if a decontamination aerosol dose flow of 0.5 ml/ft³ is administered to a 1400 ft³ room to achieve a >6 log kill and the aerosol producer operates at a flow rate of 60 ml/min, the estimated treatment time is 11.66 minutes.

B. One embodiment of the claimed invention was placed in six rooms at a municipal hospital. Touch plate results confirmed significant bacterial kill throughout the rooms treated. A six log geobacillus stearothermophilus showed at least 72 hours of no growth—an indicator that the system killed virtually all spores, bacteria, fungus and virus.

### 1 Overview

A preliminary study was performed to evaluate the capability of the disclosed decontamination system to kill high levels of bioburden in a hospital patient room.

It was found that the energized mist rapidly disinfects air and surfaces, while using only a small amount of decontamination fluid. The mist achieves inactivation of a wide range of microbiological contaminants, including vegetative bacteria, bacterial endospores, and fungi.

On Feb. 11-13, 2009 testing was conducted in 6 patient and ICU rooms at a hospital. Challenges were introduced to the environment via geobacillus stearothermophilus stainless steel coupons inoculated with geobacillus stearothermophilus spores. The purpose of using this configuration was to provide a “best representation” of the hard/non porous surface types present in a hospital environment. These test articles were subsequently incubated to evaluate the effectiveness of the inventive system.

### 2 Testing Methodology

2.1 Sample Preparation

2.1.1 Inoculated spore coupon samples were provided by Raven Laboratories in the form of geobacillus stearothermophilus 10⁶ stainless steel spore coupons. Prior to testing, each spore coupon was aseptically removed from its pouch and placed in a sterile Petri dish. Divided Petri dishes were used, placing 1 to 3 each 10⁶ spore coupons evenly spaced inside the dish. During the test at predetermined intervals, coupons were removed and placed into Raven Laboratories tryptic soy broth tubes with volumes of 5.5 ml for incubation. Dishes were placed in various locations throughout the patient's room and bathroom (if present).

2.1.2 These coupon-filled tubes were then labeled and put on ice for up to 6 hours before they were placed in the incubator (set at 55° C.) for 7 days. Tubes were then observed as noted in findings for color change in the broth which would indicate any growth and identify coupons that had spores that survived the inventive treatment.

2.2 Sample Locations

For this test, a sterile Petri dish containing 1 to 3 each of 10⁶ spore inoculated coupons was placed in each of the following locations. Room size in cubic feet is listed for each room. Distance from the inventive machine was measured and noted:
Room 7106—MICU Room (1150 ft³)
Room 2410—Patient Room (2539 ft³)
Room 2409—Patient Room (2539 ft³)
Room 535—Patient Room (2112 ft³)
Room 4104—Patient Room (1408 ft³)
Room 3112—Patient Room (1408 ft³)

2.3 Disinfection Parameters

Before decontamination, each room was hermetically sealed (e.g., the HVAC was isolated and a gasket placed around doors and windows). The treatment cycle consisted of a specific run time based on the size of the room. Samples were removed at various times during the treatment process and the final sample was taken after treatment/air scrubbing was completed. Air scrubbing took place for about 15-75 minutes immediately following fogging cycle to remove residual vapors from the air in the room, consistent with OSHA guidelines.

2.4 Sample Analysis

Spore viability on the spore coupons was determined by aseptically inoculating tryptic soy broth with the individual coupons from the field tests and incubating at 55 to 60° C. Tubes were allowed to incubate for up to one week and the tubes were observed for any evidence of growth.

### 3 Results

3.1 The test results included the sample ID number and any growth was observed for each sample. The results for the spore coupons were expressed in terms of “positive” (growth occurred) as “negative” (no growth occurred). Of 53 observations, only 9 (at longer distances) indicated “positive”. No “positives” were observed after the “air scrub” step.

3.2 Additional Findings

Measurements were also taken to determine the extent to which chemical vapors migrate into adjacent areas. The purpose of these measurements is to ensure that an area can be treated while an adjacent area is in use, without exceeding OSHA exposure limits for personnel in these adjacent areas. The short term exposure limit is ≦10 ppm and the long term exposure limit is ≦1 ppm.

Measurements of H₂O₂ vapor were taken outside each door and at wall vents of adjacent rooms. Beginning with the initiation of the fogging cycle, measurements were taken every 5 minutes.

The maximum concentrations of H₂O₂ vapor detected during the entire process at any test locations never exceeded 0.4 ppm.

### 4 Conclusions

The stainless-steel coupons typified the surfaces that must be disinfected in the hospital environment, as the vast majority of surfaces are hard and non-porous.

The samples that showed positive growth were positioned at relatively long distances from the mist-generating equipment (between 9 and 13 feet) and required the additional exposure time provided during the air scrubbing process. The inventive mist was actively killing microorganisms during the air scrubbing process, a step that in a patient room or ICU suite will always be required before re-occupancy.

Measurements taken during the trial demonstrated that there are no significant risks associated with the migration of the H₂O₂ vapor into adjacent areas.

Another feature of the system was its ease of use in a busy hospital setting. Investigators were able to prepare and treat rooms in a 1.5 to 2.0 hour cycle. Safety protocols worked easily and effectively, such that there were no excited aerosol leaks to outside areas. No odors could be detected after room treatment. No odors or noxious fumes were detected in the other hospital rooms or corridors. No residue was left at any time and surfaces did not experience wetting. The mist was completely exhausted before turning rooms over to the nursing staff.

After the treatment protocol was concluded, electronic instruments (e.g., heart rate and blood pressure monitors) performed normally.

The equipment was silent and therefore no one in the hospital was disturbed by its operation. One consequence is that rooms could be turned faster and thereby increase room occupancy rate significantly, thus increasing revenues and decreasing the housekeeping and lab staff time required.

For ease of reference, the nomenclature used herein is:

| | |
|---|---|
| 10 | Decontamination Fluid |
| 12 | Aerosol Producer |
| 14 | Uncharged Aerosol/Droplets/Mist |
| 16 | Air |
| 18 | Fan |
| 20 | Magnetic Energizer |
| 22 | Modified, Energized Droplets |
| 24 | Charging Ring |
| 26 | Further Modified, Excited Droplets |
| 28 | Target Substrate or Environment |
| 30 | Chamber |
| 32 | Controller/Microprocessor |
| 34 | Level Control Valve/Reservoir |

In summary, there exists a need for a disinfection technology that is fast, safe for human health, effective against a broad spectrum of microbial contamination including spores, and can be used in a non-contained area to provide microbial decontamination on hands, walls and surfaces. The claimed invention responds to that need.

Although particular embodiments of the invention have been described in detail for illustrative purposes, various modifications and enhancements may be made without departing from the spirit and scope of the invention. Accordingly, the invention is not to be limited except as by the appended claims.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A decontamination system for killing microorganisms on contact, the microorganisms being situated on a substrate or suspended in an ambient environment, the system comprising:
A. a source of a decontamination fluid;
B. an aerosol producer for receiving the decontamination fluid and creating an aerosol of uncharged droplets;
C. a magnetic energizer through which the aerosol passes, the energizer including a high density, uniform array magnetic field to modify the energy state of the droplets and create reactive oxygen species in or on the surface of the droplets, thereby creating modified energized droplets; and
D. a charging ring that receives the modified droplets, aligns ions therewithin and moves reactive oxygen species towards the surface of the droplets, thereby creating further modified excited droplets and allowing the further modified droplets to remain in a stable form in transit to the substrate or environment to be treated,
wherein the further modified droplets penetrating an area to be treated without dependence on airflow, the further modified droplets being similarly charged and mutually repulsive, thereby causing them to diffuse into the area be treated, so that the further modified droplets react at an ambient pressure and temperature with contaminants associated with the area to be treated and are transformed in situ into an uncharged state, thus decontaminating the substrate or environment and creating one or more benign reaction products that leave the substrate undamaged and volatilize or leave a bio-protective film on the substrate.

2. The system of claim 1, wherein the source of the decontamination fluid comprises
0.1% -10% of component A, where component A is selected from the group consisting of hydrogen peroxide, urea peroxide, and other organic peroxides;
70%-98% of component B, where component B is selected from the group consisting of water and deionized water; and
1%-10% of component C, where component C is selected from the group consisting of isopropyl alcohol, n-propyl alcohol, ethyl alcohol, and protic/aprotic polar solvents.

3. The system of claim 2, further including an antimicrobial additive that may leave a bioprotective residue on the substrate after decontamination.

4. The system of claim 2, further including boric acid for treating a substrate or environment that includes an infestation of bed bugs, lice and like pests.

5. The system of claim 1, wherein the source of the decontamination fluid comprises a source of an excitable species selected from the group consisting of hydrogen peroxide, urea peroxide and other organic peroxide complexes.

6. The system of claim 1, wherein the source of the decontamination fluid further comprises 0.05-3% of a promoting specie selected from the group consisting of ethylenediaminetetraacetic acid, chelated metal ions, ozone, and mixtures thereof.

7. The system of claim 1, wherein the source of the decontamination fluid further comprises a promoting specie selected from the group consisting of an alcohol, an enzyme, a fatty acid, an acid, a chelating agent, and mixtures thereof.

8. The system of claim 1 wherein substantially all of the excited droplets react with contaminants associated with the substrate or environment.

9. The system of claim 1, further including a source of thermal energy for raising the temperature of the decontamination solution.

10. The system of claim 1, wherein the aerosol producer and the magnetic energizer are disposed proximally, so that the energizer influences the droplets in the aerosol of the decontamination fluid as they leave the aerosol producer.

11. The system of claim 1, wherein the magnetic energizer is located remotely from the aerosol producer.

12. The system of claim 1, wherein the system further includes a chamber into which is placed an object or member to be decontaminated, into which the excited droplets of the decontamination fluid are directed.

13. The system of claim 1, wherein the system operates in an open environment.

14. The system of claim 1, wherein the magnetic energizer includes an apparatus selected from the group consisting of a Helmholtz coil, a Maxwell coil and other such coil arrays that are energized by a direct or alternating electric current.

15. The system of claim 1, wherein the components are arranged in a sequence selected from the group consisting of
A, B, C, D;
A, C, D, B; and
A, C, B, D.

16. The system of claim 1, further including a fan.

17. The system of claim 1, further including a microprocessor.

18. A method for performing decontamination, comprising the steps of
I. magnetically exciting droplets of a decontamination fluid, the excited droplets including droplets that transport reactive oxygen species; and
II. introducing the excited droplets into an environment or to a substrate to be decontaminated.

19. The method of claim 18, wherein step II further includes introducing the excited droplets into an enclosure wherein decontamination occurs at least partially within the enclosure.

20. The method of claim 18, wherein step II occurs within an enclosed chamber.

21. The method of claim 18, wherein step II occurs in an open space.

22. The method of claim 18, wherein step I includes the steps of providing a decontamination system with
A. a source of a decontamination fluid being selected from the group consisting of a gas, a liquid, and mixtures thereof,
B. an aerosol producer selected from the group consisting of a nebulizer, a fogger, and a sprayer, the aerosol producer receiving the decontamination fluid at a flow rate between 3 and 200 milliliters per minute and creating an aerosol of small substantially uncharged droplets;
C. a magnetic energizer through which the aerosol passes, the energizer including a high density, uniform array magnetic field to create reactive oxygen species within and on the surface of the droplets during a dwell time of between 0.25-3 seconds, thereby creating energized droplets in which 20-80% of the hydrogen peroxide in the droplets is converted into reactive oxygen species; and
D. a charging ring that receives the energized droplets, aligns ions within the energized droplets and moves reactive oxygen species towards the surface of the droplets, thereby allowing most of the further modified droplets to remain in a relatively stable form in which they may be held in transit to the substrate or environment to be treated,
wherein the further modified droplets have an average particle size of 1-100 microns to facilitate penetration into an area to be treated, so that dispersion is relatively widespread without significant dependence on airflow, the further modified droplets being similarly charged and mutually repulsive, thereby causing them to diffuse into an ambient environment or onto a substrate to be treated, so that the further modified droplets react at an ambient pressure and temperature with contaminants associated with the substrate or environment and are transformed in situ into an uncharged state, thus decontaminating the substrate or environment and creating one or more
benign reaction products that leave the substrate undamaged and volatilize or leave a bio-protective film on the substrate.

23. A method for decontaminating a microorganism situated on a substrate or suspended in an ambient environment, comprising the steps of:
I. providing a source of a decontamination fluid;
II. introducing an aerosol producer that receives the decontamination fluid and creates an aerosol of uncharged droplets;
III. ducting the aerosol of uncharged droplets through a magnetic energizer, thereby subjecting them to a high density, uniform array magnetic field to modify the energy state of the droplets and create reactive oxygen species in or on the surface of the droplets, thereby creating modified droplets; and
IV. passing the modified droplets through a charging ring that aligns ions therewithin and moves reactive oxygen species towards the surface of the droplets, thereby creating further modified droplets and allowing the further modified droplets to remain stable in transit to the substrate or environment to be treated, the further modified droplets being similarly charged and mutually repulsive, thereby causing them to diffuse into the area be treated,
whereby the further modified droplets penetrate an area to be treated without dependence on airflow, so that the further modified droplets react at an ambient pressure and temperature with contaminants associated with the area to be treated and are transformed in situ into an uncharged state, thus decontaminating the substrate or environment and creating one or more benign reaction products that leave the substrate undamaged and volatilize or leave a bio-protective film on the substrate.

24. A decontamination system for killing microorganisms on contact, the microorganisms being situated on a substrate or suspended in an ambient environment, the system comprising:
A. a source of a decontamination fluid being selected from the group consisting of a gas, a liquid, and mixtures thereof,
B. an aerosol producer selected from the group consisting of a nebulizer, a fogger, and a sprayer, the aerosol producer receiving decontamination fluid from the source, transported by a carrier at a flow rate between 3 and 200 milliliters per minute, the decontamination fluid and creating an aerosol of small substantially uncharged droplets suspended in air, the droplets having a high surface area to volume ratio;
C. a magnetic energizer through which the aerosol passes, the energizer including a high density, uniform array magnetic field to produce from the uncharged droplets modified droplets which contain reactive oxygen species within and on the surface of the modified droplets during a dwell time of between 0.25-3 seconds; and
D. a charging ring that receives the modified droplets, aligns ions within the modified droplets and moves reactive oxygen species towards the surface of the modified droplets, thereby forming further modified droplets that remain in a relatively stable form while in transit to the substrate or environment to be treated,
wherein the further modified droplets have an average particle size of 1-100 microns to facilitate penetration into an area to be treated, so that dispersion is relatively widespread without significant dependence on airflow, the further modified droplets being similarly charged and mutually repulsive, thereby causing them to diffuse into an ambient environment or onto a substrate to be treated, so that the further modified droplets react at an ambient pressure and temperature with contaminants associated with the substrate or environment and are transformed in situ into an uncharged state, thus decontaminating the substrate or environment and creating one or more benign reaction products that leave the substrate undamaged and volatilize or leave a bio-protective film on the substrate.
